(19) United States
(12) Patent Application Publication
(10) Pub. No.: US 2010/0197584 A1
(43) Pub. Date: Aug. 5, 2010

(54) USE OF CURCUMIN TO BLOCK BRAIN TUMOR FORMATION IN MICE

(75) Inventors: Probal Banerjee, Staten Island, NY (US); Krishnaswami Sambasivan Raja, Staten Island, NY (US)

Correspondence Address: Fish & Richardson P.C. / CUNY;Research Foundation of the City University of New, P.O. Box 1022, Minneapolis, MN, 55440-1022, US

(73) Assignee: Research Foundations of the City University of- New York, New Yorlk, NY (US)

(21) Appl. No.: 12/670,685

(22) PCT Filed: Jul. 25, 2008
(86) PCT No.: PCT/US08/09053

§ 371 Date: Jan. 27, 2010

Related U.S. Application Data
(60) Provisional application No. 60/962,261, filed on Jul. 27, 2007.

(51) Int. Cl. A61K 31/12; A61P 35/00; A61P 35/04; A61K 38/22; A61P 3/02

(52) U.S. Cl. 514/12; 514/679

(57) ABSTRACT

The present invention provides compositions and methods of using curcumin or curcumin derivatives or analogs to activate the pro-apoptotic enzymes caspase-3/7 in cancer cells. The present invention also provides formulations of curcumin or derivatives or analogs with increased solubility or improved bioavailability. The formulations may be administered to a subject such that high concentrations of therapeutically effective curcumin compounds resuit in the subject's bloodstream. The invention thus involves the use of curcumin or curcumin derivatives or analogs to diminish cancer cell growth, decrease tumor size, prevent tumor formation, and Curcumin Carrier to reduce or prevent cancer or tumor cell invasion or metastasis into a tissue, e.g., into the nervous System and especially the brain, of a subject. The instant invention may be used prophylactically to prevent tumor formation or metastasis, as a monotherapy to treat existing tumors, after surgery to prevent recurrence of tumors or in conjunction with conventional cancer therapies to improve patient prognosis and reduce side-effects.

### BACKGROUND OF THE INVENTION

Curcuma longa is a tropical plant native to south and southeast tropical Asia. Derived from the root of the plant Curcuma longa, a polyphenol, termed turmeric, has been used for treatment of different inflammatory diseases and has been described in Ayurveda and in traditional Chinese medicine for thousands of years (Shishodia, et al. Ann NY Acad. Sci., 2005. 1056(1): p. 206-217). Isolated from turmeric and known to give curry its yellow color, curcumin has been known to possess many pharmacologic properties. It has been proven to exhibit remarkable anticancer, anti-inflammatory and antioxidant properties (Phan, T.-T., et al. Trauma, 2001. 51: p. 927-931). Chemopreventive and growth inhibitory activities against many tumor cell lines have been reported (Deeb, D., et al. Mol Cancer Ther, 2004. 3(7): p. 803-812). Specifically, curcumin shows anticarcinogenic activity in prostate cancer (Hong, J. H., et al., Prostate Cancer Prostatic Dis, 2006. 9(2): p. 147-152), breast cancer (Bachmeier, B. E., et al. Cellular Physiology and Biochemistry, 2007. 19(1-4): p. 137-152) and colon cancer (Chen, A., et al. Oncogene, 2005. 25(2): p. 278-287).

Epidemiological investigations have shown a significant difference in the incidence of cancers among ethnic groups having different lifestyles and who have been exposed to various environmental factors. It has been estimated that more than two-thirds of human cancers, contributed by mutations in multiple genes, could be prevented by modification of lifestyle such as dietary modification (Sarkar, F. H., et al. Mutation Research/Fundamental and Molecular Mechanisms of Mutagenesis, 2004. 555(1-2): p. 53-64). Curcumin, a member of the Zingiberaceae family, stands as a candidate for this dietary modification of lifestyle.

A recent study suggests that curcumin reduces tumor growth in an orthotopic murine model of ovarian cancer (Lin, Y. G., et al. Clin Cancer Res. 2007

Jun. 1; 13(11): pp. 3423-30). Other studies report that curcumin induces apoptosis in melanoma cell lines (Siwak, D. R., et al. Cancer 2005 Aug. 15; 104(4): pp. 879-90) and human pancreatic carcinoma cell lines (Li, L., et al. Cancer 2004 Nov. 15; 101(10): pp. 2351-62) and has antiproliferative effects on several breast tumor cell lines (Mehta, K., et al. Anticancer Drugs 1997 June; 8(5): pp. 470-81). Clinical studies exploring the effect of curcumin on various cancers are ongoing (see, e.g., Hsu, C. H., et al. Adv Exp Med. Biol. 2007; pp. 595:471-80; Shishodia, S., et al. Curr Probl Cancer. 2007 July-August; 31(4): pp. 243-305 for reviews of clinical studies).

To date, there are only two reports on the effect of curcumin on cancer cells of neural origin, which demonstrate that this compound triggers apoptosis in human and rat glioma cells in vitro (Karmakar, S., Banik, N. L., Patel, S. J., and Ray, S. K., Neuroscience Letters, 2006. 407(1): p. 53-58; Dhandapani, K. M., Mahesh, V. B., and Brann, D. W., J. Neurochem., 2007. 102: p. 522-538). However, no studies report the use of curcumin in blocking brain tumor formation in vivo. Additionally, one study has described the use of curcumin in eliminating cancer, e.g., melanoma-mediated cancer, in vitro (Siwak, D. R., Shishodia, S., Aggarwal, B. B., and Kurzrock, R., Cancer 2005. 104: p. 879-890). But no studies report the use of curcumin in eliminating cancer, e.g., melanoma-mediated cancer, in vivo. This invention demonstrates both chemopreventive as well as anti-tumor properties of curcumin in the mouse nervous system and in infiltrating melanoma in mouse brain.

The common therapy available currently for brain tumor involves surgery followed by radiation and chemotherapy, both of which are involved with many adverse side effects. In addition, conventional therapies like radiation have limited pediatric use due to the potential damage to the developing brain. Despite all advancements, survival rates from brain tumors are well below 50% and the average survival period is less than two years. One reason for this poor outcome is the lack of safe agents to eliminate residual brain tumor cells after surgical resection of the lump and the high incidence of metastasis of non-brain tumors into the brain.

It would be beneficial to have more effective treatments for cancers and tumors of all kinds, and particularly for nervous system associated tumors, e.g., brain and spinal tumors, comprising cell types of any origin.

### SUMMARY OF THE INVENTION

The present invention helps solve the problems described above by providing improved compositions comprising curcumin, or an analog or derivative thereof (“curcumin compound”). Also provided are methods for administering such compositions to a subject in need of treatment so that higher concentrations of a therapeutically effective curcumin compound is achieved in the subject's bloodstream than those which have been achieved previously, e.g., by oral administration.

In some embodiments, the present invention provides methods for diminishing cancer cell growth, decreasing tumor size, inhibiting or preventing tumor formation, and inhibiting or preventing cancer or tumor cell invasion or metastasis into a tissue of a subject. Each of the above methods comprises the step of administering a composition comprising a curcumin compound. In particular embodiments, the cancer is associated with nervous system tissue, e.g., brain tumors. In certain embodiments, the brain tumor is a metastasis from a primary tumor.

In other embodiments, the present invention provides methods for or inhibiting or preventing the recurrence of tumors after surgery, radiation or chemotherapy, improving cancer patient prognosis, increasing remission time, and increasing the survival time in a subject with cancer, each method comprising the step of administering a composition comprising a curcumin compound. In particular embodiments, the cancer is associated with nervous system tissue, e.g., brain tumors. In certain embodiments, the brain tumor is a metastasis from a primary tumor.

The present invention also provides methods of improving and/or diminishing the side effects of traditional methods of cancer therapy or treating cancers that are ineffectively treated by traditional methods, each method comprising the step of administering a composition comprising a curcumin compound. In particular embodiments, the cancer is associated with nervous system tissue, e.g., brain tumors. In certain embodiments, the brain tumor is a metastasis from a primary tumor.

The present invention further provides methods for decreasing, inhibiting or preventing angiogenesis in a subject. In particular embodiments, the invention provides a method for decreasing vascularization, or inhibiting or preventing neovascularization of a tumor in the body, each method comprising the step of administering a composition comprising a curcumin compound. In particular embodiments, the tumor is associated with nervous system tissue, e.g., a brain tumor. In certain embodiments, the brain tumor is a metastasis from a primary tumor.

In some embodiments, the present invention provides compositions comprising one or more curcumin compounds with increased solubility or improved bioavailability, including formulations wherein the active compound passes the blood-brain barrier. In certain embodiments, a curcumin composition of the invention further comprises at least one therapeutically active agent in addition to a curcumin compound. In certain embodiments, a curcumin composition of the invention comprises DMSO. In certain embodiments, the curcumin composition is in a dosage form that is injectable. In a particular embodiment, the dosage form is suitable for injection into a vein.

### BRIEF DESCRIPTION OF THE DRAWINGS

FIG. 1 is a series of graphs showing caspase 3/7 activity in B16F10 mouse melanoma cells in the presence and absence of curcumin treatment for 48 h (1A) or 96 h (1B).

FIG. 2 is a series of graphs showing caspase 3/7 activity in N18 mouse neuroblastoma cells in the presence and absence of curcumin treatment for 48 h (2A) or 96 h (2B).

FIG. 3 is a series of graphs showing caspase 3/7 activity in HOG human oligodendroglioma cells in the presence and absence of curcumin treatment for 48 h (3A) or 96 h (3B).

FIG. 4 is a graph showing caspase 3/7 activity in GL261 mouse glioma cells in the presence and absence of curcumin treatment for 48 h.

FIG. 5 is a graph showing caspase 3/7 activity in A549 human lung carcinoma cells in the presence and absence of curcumin treatment for 48 h.

FIG. 6 is a series of graphs showing B16F10 mouse melanoma (6A), GL261 mouse glioma (6B), N18 mouse neuroblastoma (6C), A549 human lung carcinoma (6D) and HOG human oligodendroglioma (6E) cell viability (determined by MTT assay) in the presence and absence of curcumin treatment for 48 h (top panel) or 96 h (bottom panel). The y-axis represents cell viability expressed as % of DMSO control.

FIG. 7 is a series of HPLC traces showing the presence of curcumin in the brain of a mouse 15 minutes (7B), 30 minutes (7C) or 2 hours (7D) after injection of curcumin into a tail vein. No curcumin is observed in the brain of a mouse 15 minutes after injection of carrier into a tail vein (7A).

FIG. 8 contains images of hematoxylin & eosin double stained brain tissue sections from mice injected with B16F10 mouse melanoma cells and then subsequently injected with carrier control or curcumin solution. (8A) is a tissue section from a carrier control injected mouse at 1× magnification. (8B) is a tissue section from a curcumin injected mouse at 1× magnification. (8C) is a tissue section from a carrier control injected mouse at 10× magnification.

FIG. 9 shows protein levels of signaling molecules that regulate tumor cell viability in the presence or absence of curcumin treatment for 48 h or 96 h. The numbers below the protein bands indicate their respective intensities expressed as % of the carrier-treated control for a specific set of samples (i.e. 48 h or 96 h).

FIG. 10 is a graph showing the viability of normal (non-tumor) brain cells following carrier, curcumin or H₂O₂ treatment.

FIG. 11 shows the formation of dark melatonin expressing tumors in carrier treated, but not curcumin treated, whole-mount brains following intracranial injections. 11A and 11B represent two identical, yet independent, experiments.

### DETAILED DESCRIPTION OF THE INVENTION

### Definitions

The term “cancer” refers to all types of cancer, neoplasms, or tumors, whether or not solid or single cells, found in mammals, including, e.g., hematopoietic cancer, carcinomas, melanomas, sarcomas and non-malignant tumors.

Commercial curcumin includes three major components: curcumin, desmethoxycurcumin, and bisdesmethoxycurcumin, which are often referred to as “curcuminoids.” As used herein, “curcumin” is defined to include any one or more of these three major components of commercial curcumin, and a “curcumin compound” is defined as curcumin or any active derivative or analog thereof. This includes natural and synthetic derivatives of curcumin and curcuminoids, and includes any combination of more than one curcuminoid or derivative or analog of curcumin. Analogs of curcumin and curcuminoids are well-known in the art, and include, e.g., those derivatives or analogs disclosed in U.S. Patent Application Publication 20020019382, Kumar, S., et al, Nucleic Acids Symp Ser. 2000; (44): pp. 75-6; Mishra, S., et al. Nucleic Acids Res Suppl. 2002; (2): pp. 277-8; Dinkova-Kostova, A. T., Mini Rev Med. Chem. 2002 December; 2(6): pp. 595-610; Ohtsu, H., et al. J Med. Chem. 2002 Nov. 7; 45(23): pp. 5037-42; Ishida, J., et al. Bioorg Med. Chem. 2002 November; 10(11): pp. 3481-7).

The term “derivative” or “analog” as used herein refers to a natural or synthetic compound which is structurally similar to curcumin and which has at least one biological activity in common with curcumin.

The term “subject” as used herein refers to an animal that has been the object of treatment, observation or experiment. The animal may be a mammal. In some embodiments, the subject is a human.

The term “subject in need thereof” as used herein refers to a subject who is in need of treatment or prophylaxis as determined by one of skill in the art, for example, a researcher, veterinarian, medical doctor or other clinician.

The term “therapeutically effective amount” as used herein means an amount of an active compound in a composition that will elicit a biological or medical response in a tissue, system, subject, or human that is being sought by the researcher, veterinarian, medical doctor or other clinician, including, e.g., inhibiting or blocking tumor formation or metastases, reducing tumor size or reducing or inhibiting angiogenesis.

The term “prophylactically effective amount” as used herein means an amount of an active compound in a composition that will elicit a biological or medical response in a tissue, system, subject, or human that is of a preventative nature or relating to a prophylactic treatment which is being sought by the researcher, veterinarian, medical doctor or other clinician, including, e.g., prevention of tumor formation, metastases or angiogenesis.

As used herein, “pharmaceutically acceptable carrier” or “pharmaceutical carrier” includes any and all solvents, dispersion media, coatings, surfactants, antioxidants, preservatives (i.e., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, e.g., Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The term “prolonged,” “prolonged administration,” or “prolonged treatment” as used herein, means administration of a compound, preferably curcumin (as defined herein), as either a series of boluses or otherwise by continuous administration. Prolonged treatment or administration may last for an extended period of time, including administration or treatment for up to one week, ten days, two weeks, one month, three months, six months, one year, two years, three years, indefinitely, or until the treatment has no further beneficial effect.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are each incorporated herein by reference in its entirety. In case of conflict, the present specification, including definitions, will control. The materials, methods, and examples are illustrative only and not intended to be limiting.

The present invention solves the problem of low survival rate from brain cancer by providing methods and compositions for eradicating residual brain tumors, inhibiting neoplastic growth, decreasing tumor invasion and metastasis, and increasing mean patient survival times and remission time by treatment with a curcumin compound.

The present invention thus provides compositions of and methods using a curcumin compound to treat tumor cells, including brain tumor cells, in vitro or in vivo, to diminish cancer cell growth or decrease tumor size. When administered to a subject, the curcumin compound is able to cross the blood-brain barrier (see e.g. Example 3) and is thus suitable for administration using any appropriate methods known in the art, including but not limited to intravenous, oral, transdermal and transmucosal administration. In certain embodiments, the compositions and methods of this invention are useful in methods for improving cancer patient prognosis.

In some embodiments, the disclosed methods comprise the step of administering a curcumin compound or a curcumin composition prophylactically, e.g., to prevent tumor formation or to reduce tumor growth or metastases. In particular embodiments, the tumor is one of the central nervous system, such as a brain tumor. In other embodiments, the disclosed methods may be used to treat subjects after tumor resection surgery to prevent the reappearance of cancer. The disclosed methods and compositions may be used in conjunction with, or as an alternative to, conventional therapy methods including tumor resection surgery, radiation, and chemotherapy and are useful to reduce side effects of these therapies. In some embodiments, the disclosed methods and compositions are used instead of radiation and chemotherapy to avoid the adverse side-effects of these therapies. In some embodiments, a curcumin compound or composition will be used to treat a subject who is in remission from cancer to increase remission or survival time by about 1 month, 3 months, 6 months, 9 months, 1 year, 2 years, 3 years, 5 years or 10 years. In some embodiments, a curcumin compound or composition may be administered to a subject to prevent or inhibit the recurrence of tumors, e.g., of a brain tumor.

A large number of brain tumors are caused by metastatic invasion of cancer cells, including melanoma, from other parts of the body (see, e.g., Denkins, et al. Neuro Oncol. 2004 April; 6(2): pp. 154-65). Prognosis for such patients is grim (Prados, M. D. and Wilson, C. B., in Cancer Medicine, Third Edition. Philadelphia: Lea & Febiger, 1993 pp. 1080-1119; Sawaya et al. J. Neurooncol. 1996 27: pp. 269-277; Soffietti et al., J. Neurol. 2002 249: pp. 1357-1369). The present invention provides a means of and methods for combating the spread of melanoma or other cancer cells and preventing, decreasing or blocking metastasis to tissues, including the brain; inhibiting neoplastic growth; decreasing tumor invasion and metastasis; and increasing mean patient survival time and remission time.

In other embodiments, a composition comprising a curcumin compound may be used to diminish the development of cancer in vivo in peripheral regions of the subject. For example, as exemplified herein, injection of B16F10 mouse melanoma cells in the neck region of mice caused cancerous growth and debilitation in the absence of curcumin treatment, whereas curcumin-treated mice did not develop cancer. See e.g. Example 4. In some embodiments, the invention provides a method to inhibit or block the formation and/or growth of breast, ovary, colon, lung, central nervous system, kidney and prostate cancers or melanoma.

The present invention also provides curcumin compositions and methods using a curcumin compound or composition to activate the pro-apoptotic enzymes caspase-3/7 in cancer cells or cell lines. Non-limiting examples of cells or cell lines that are responsive to curcumin compounds include B16F10 (mouse melanoma), N18 (mouse neuroblastoma), GL261 (mouse glioma), A549 (human lung carcinoma) and HOG (human oligodendroglioma) cells. See e.g. Example 2.

In some embodiments, the compounds, compositions and methods of the present invention are used to treat cancer in a subject. The term “cancer” as used herein refers to all types of cancer or neoplasm or malignant or non-malignant tumors found in mammals, including leukemia, carcinomas and sarcomas.

Non-limiting examples of cancers which may be treated according to the invention include cancer of the brain, breast, prostate, cervix, colon, head and neck, kidney, lung, small and non-small cell lung, melanoma, mesothelioma, ovary, sarcoma, stomach, uterus and medulloblastoma.

Leukemias are malignant diseases of the blood-forming organs and are generally characterized by a distorted proliferation and development of leukocytes and their precursors in the blood and bone marrow. Leukemia diseases include, for example, acute non-lymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Carcinomas are malignant new growths made up of epithelial cells tending to infiltrate the surrounding tissues and give rise to metastases. Exemplary carcinomas include acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kapos's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

In some embodiments, the methods and compounds of the present invention are used to treat malignant or non-malignant tumors of the central nervous system (CNS). Gliomas, metastases, meningiomas, pituitary adenomas, and acoustic neuromas account for the majority of brain tumors in humans.

Examples of glial tumors (gliomas) include astrocytoma, pilocytic astrocytoma, low-grade astrocytoma, anaplastic astrocytoma, glioblastoma multiforme, brain stem glioma, ependymoma, subependymoma, ganglioneuroma, mixed glioma, oligodendroglioma, and optic nerve glioma.

Examples of non-glial tumors include acoustic neuroma, chordoma, CNS lymphoma, craniopharyngioma, hemangioblastoma, medulloblastoma, meningioma, pineal tumors, pituitary tumors, primitive neuroectodermal tumors (PNET), rhabdoid tumors, and schwannoma.

Tumors that affect the cranial nerves include gliomas of the optic nerve, neurofibromas of 8th cranial nerve, neurofibromas of 5th cranial nerve.

Benign tumors include arachnoid, dermoid, epidermoid, colloid, and neuroepithelial cysts and any other slow growing tumor.

While primary brain tumors, like those described above, originate in the brain itself, metastatic brain tumors (secondary brain tumors that begin as cancer in another part of the body) are the most common brain tumors. Cerebral metastases can spread from primary cancers including, but not limited to, cancers originating in the lung, skin (melanoma), kidney, colon and breast. In certain embodiments, the present invention encompasses treating any primary cancer that is capable of metastasizing to the brain or preventing or reducing metastasis of such cancers to the brain. See e.g., Example 5 and Example 8.

In some embodiments, the invention will be used to decrease or inhibit angiogenesis in a subject. See, e.g., Example 6. Positive markers of angiogenesis are well-known in the art and include, but are not limited to, increased FGF, VEGF, Ang1, Ang2, Tie1, Tie2, and D114 expression and matrix metalloproteinase (MMP) activity.

### Pharmaceutical Compositions and Administration

A curcumin compound may be administered to a subject as a pharmaceutically acceptable salt or prodrug in the presence of a pharmaceutically acceptable carrier or diluent, for any of the indications or modes of administration as described in detail herein. The active materials may be administered by any appropriate route, for example, orally, parenterally, enterally, intravenously, intradermally, subcutaneously, transdermally, intranasally, intramuscularly, intraperitoneally, mucosally, or topically, in liquid or solid form including by aerosol particle delivery to the lungs as described, e.g., in U.S. Patent Application Publications US 2005/0181036 and US 2003/0149113; and U.S. Pat. Nos. 6,613,308; 6,673,843; 6,664,272; 5,401,777; 5,543,158; 5,641,515; and 5,399,363.

Curcumin compositions of the present invention may be used alone or in combination with other therapeutic agents. Thus, a curcumin compound may be combined with at least one other therapeutic agent in a single formulation, or separate formulations may be administered to the subject at the same or at different times (e.g., co-administration or alternating or independently intermittent administrations), as selected by the skilled practitioner under the particular circumstances.

Thus, in some embodiments, the curcumin compound is administered with at least one other agent including, but not limited to, anticancer, antioxidant, anti-inflammatory, apoptotic agents, hormones, growth factors, nutrients, and diagnostic agents. Examples of antitumor drugs that may be administered with curcumin or a curcumin derivative include, for example. amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, gliadel implants, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, streptozocin, tegafur-uracil, temozolomide, teniposide, thiotepa, tioguanine, topotecan, treosulfan, vinblastine, vincristine, vindesine, and vinorelbine.

Depending upon the particular curcumin compound, the composition (e.g., formulation) and its selected route of administration, the amount of curcumin compound administered to a subject may vary. For example, in certain embodiments, the curcumin compound may be formulated such that the effective concentration of curcumin (or derivative or analog thereof) that is delivered in a single dosage form to a target cell is from about 0.1 μM-200 μM. The concentration of active compound in the drug composition will depend on absorption, distribution, metabolism and excretion rates of the drug as well as other factors known to those of skill in the art. It is to be noted that dosage values may also vary with the severity of the condition to be alleviated and the subject to be treated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

In one embodiment of the invention, prolonged treatment may be accomplished by continuous administration of an effective amount of a curcumin compound, e.g. via a minipump, an implantable slow-release form of the curcumin compound, or intravenous drip administration. Alternatively, prolonged treatment may be accomplished by repeatedly administering an amount of a curcumin compound at a dose level and dosage interval such that the curcumin compound concentration in the serum, cell or tissue of interest never drops below the concentration that is required for the selected method, e.g., to diminish cancer cell growth, decrease tumor size, or prevent tumor formation and tumor cell invasion. See, e.g. Example 4, Example 5, and Example 8. Methods of determining the pharmacokinetic profile of a particular curcumin compound are well-known in the art and may be used in empirically determining the precise dose and dosage interval to maintain an effective concentration. Repeated administration may be accomplished, e.g. by administration once every 12 hours, once a day, every other day, twice a week, once a week, every other week, once a month, once every three months, once every six months, or once a year.

In one embodiment of the invention, the dose range of the curcumin compound (including a derivative or analog thereof) will be an amount that results or achieves a blood or plasma concentration of about 5 μM, 10 μM, 15 μM, 20 μM, 25 μM, 30 μM, 35 μM, 40 μM, 45 μM, 50 μM, 70 μM, 80 μM, 100 μM or any range there between.

In specific embodiments, the active agent is curcumin (molecular weight 368.39) and a therapeutically effective amount of curcumin is the amount, when administered to a subject, that results in a blood or plasma concentration of curcumin in the range of about 5 μM to about 100 μM, 15 μM to about 85 μM, 30 μM to about 80 μM, 20 μM to about 80 μM, 30 μM to about 70 μM, 25 μM to about 75 μM, 25 μM to about 60 μM or 25 μM to about 50 μM. Curcumin analogs and derivatives may have different efficacy and toxicity profiles than curcumin, and may be tailored to the individual needs of the subject. One of skill in the art is able to empirically determine the therapeutically effective blood or plasma concentration of such curcumin analogs and derivatives, which may be lower or higher than the therapeutically effective curcumin concentration. One of skill in the art is able to determine the blood or plasma levels of curcumin or its analogs or derivatives using standard procedures known in the art to measure levels of compounds in the blood or plasma.

A preferred dose of curcumin compound will be in the range of from about 1 to 75 mg/kg, preferably 1 to 20 mg/kg, of body weight per day, more generally 0.1 to about 100 mg per kilogram body weight of the recipient per day. The effective dosage range of the pharmaceutically acceptable derivatives can be calculated based on the weight of curcumin or curcumin analog or derivative to be delivered.

The compounds are conveniently administered in any suitable dosage form, including but not limited to one unit containing 1 mg to 8000 mg, preferably 100 mg to 1500 mg of active ingredient per unit dosage form. An oral dosage of 50 mg to 1000 mg is usually convenient. An intravenous dosage of 50 mg to 1000 mg is usually convenient.

Curcumin has low solubility in water. To increase its solubility, curcumin may be dissolved in DMSO in sterile phosphate buffered saline (PBS). See, e.g., Example 1. We have found that curcumin, and many curcumin analogs and derivatives, shows surprisingly effective bioactivity when administered to a subject in a formulation comprising DMSO. In some embodiments, a curcumin compound is formulated with DMSO at a concentration of from about 1% to 20%. In some embodiments, a curcumin compound is formulated with DMSO at a concentration of from about 1% to 15%. In other embodiments, the concentration of DMSO is from about 2% to 10%. In certain particular embodiments, the concentration of DMSO is from about 15%-17% or 15%-20%.

In certain particular embodiments, the concentration of DMSO is from about 3% to 5%. When 200 μl of 667 μM curcumin solution comprising 3% DMSO is injected into a mouse (approximately 4 ml of body fluid), the final concentration of DMSO is expected to be about 0.15%. See, e.g. Example 4 and Example 5. When 5 μl of a 3 mM curcumin solution made with 15% DMSO is injected intracranially (average brain volume of 400 μl), the final concentration of DMSO is expected to be about 0.187%. See, e.g. Example 8. Thus, in certain embodiments of the invention, a curcumin composition comprising DMSO as a solublizing agent is administered in a dosage unit that achieves or results in a final blood or plasma DMSO concentration of about 0.05% to 0.5%, 0.05% to 0.25%, 0.1% to 0.4%, 0.1% to 0.3%, 0.1% to 0.25%, 0.1% to 0.2%, 0.15% to 0.30%, 0.15% to 0.25% or 0.15% to 0.2%.

A large body of literature already exists on the human toxicology of DMSO (see, e.g., Brobyn, R. D. Ann N Y Acad. Sci. 1975; 243: pp. 497-506; Santon, N. C., et al. Biochemical Pharmacology, 2003; 65: pp. 1035-1041). Studies and human trials using relatively higher amounts of DMSO (0.5%) have shown that it is a safe vehicle for drug formulation. Therefore, the DMSO concentration of such formulations of curcumin and curcumin derivatives are expected to have no adverse effect, even in humans.

Curcumin solubility may be increased by using nanoparticle-based formulations. Polymeric nanoparticle encapsulated formulations of curcumin—nanocurcumin—utilizing the micellar aggregates of cross-linked and random copolymers of N-isopropylacrylamide (NIPAAM), with N-vinyl-2-pyrrolidone (VP) and poly(ethyleneglycol)monoacrylate (PEG-A) are known to be readily dispersed in aqueous media and are likely to have increased bioavailability (Bisht, S., et al. J Nanobiology 2007 Apr. 17; 5:3).

In some embodiments, a curcumin compound, or composition comprising a curcumin compound, may be administered intravenously. See, e.g. Example 4 and Example 5. If administered intravenously, preferred carriers are physiological saline or phosphate buffered saline (PBS). The carrier may also comprise DMSO and/or one or more other solubilizing agents. We have shown that DMSO is an effective solubilizing agent and is useful in a formulation for intravenous administration. Other solubilizing agents are well known to the art (see, e.g., Remington's Pharmaceutical Sciences, 18th Ed. Mack Printing Company, 1990, pp. 1289-1329) and may be selected by the skilled worker in consideration of factors including the desired administration route.

Curcumin compounds and compositions comprising them may be administered orally according to a method of the invention. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible bind agents, and/or adjuvant materials can be included as part of the composition. Oral curcumin compositions formulated to enhance curcumin bioavailability or solubility may also be used in any one of the methods of the invention (see, e.g., WO 2008/051474; WO 2008/045534; US 2007/0270464; WO 2006/129323; WO 2006/022012).

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The curcumin compounds or their pharmaceutically acceptable derivative or salts thereof, and compositions comprising them, may be administered as a component of an elixir, suspension, syrup, wafer, chewing gum or the like. A syrup may contain, in addition to the active compounds, sucrose as a sweetening agent and certain preservatives, dyes and colorings and flavors.

The curcumin compounds or their pharmaceutically acceptable derivative or salts thereof may also be mixed with other active materials that do not impair the desired action. Solutions or suspensions used for parental, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

The active compounds may be formulated with a lipid vehicle. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. One of ordinary skill in the art would be familiar with the range of techniques that can be employed for dispersing a drug in a lipid vehicle. For example, the curcumin may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes (see, e.g., WO2005/020958).

Liposomal suspensions may also be used as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension. Lipsomal curcumin formulations are known (US Patent Application Publication 2006/0067998).

Additional formulations which are suitable for other modes of administration include suppositories. Suppositories are solid dosage forms for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Controlled Release Formulations

Curcumin or a curcumin derivative can be administered as a controlled release formulation. The field of biodegradable polymers has developed rapidly since the synthesis and biodegradability of polylactic acid was reported by Kulkarni et al., in 1966 (Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 (polyanhydrides), U.S. Pat. No. 4,767,628 (polylactide, polylactide-co-glycolide acid), and U.S. Pat. No. 4,530,840, et al. (polylactide, polyglycolide, and copolymers). See also U.S. Pat. No. 5,626,863 which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and WO 97/05185 directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 discloses a method for forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

U.S. Pat. No. 5,728,402 describes a controlled release formulation that includes an internal phase which comprises the active drug, its salt or prodrug, in admixture with a hydrogel forming agent, and an external phase which comprises a coating which resists dissolution in the stomach. U.S. Pat. Nos. 5,736,159 and 5,558,879 discloses a controlled release formulation for drugs with little water solubility in which a passageway is formed in situ. U.S. Pat. No. 5,567,441 discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery.

WO2007/070983 discloses other formulations for transdermal delivery of pharmaceutical agents. U.S. Pat. No. 5,505,962 describes a controlled release pharmaceutical formulation.

### Curcumin Derivatives, Analogs and Prodrugs

A variety of curcumin derivatives and analogs are known in the art and may be used in the present invention (see, e.g., WO 2007/051314; US 2006/0276536). Such derivatives may have increased solubility or potency. Examples of curcumin derivatives include bis(arylmethylidene)acetone (WO 2007/000998), desmethoxy curcumin and bisdesmethoxy curcumin (WO 2006/117077). Other curcumin analogs that may be used include dihydrocurcumin, tetrahydrocurcumin, hexahydrocurcumin, dihydroxytetrahydrocurcumin, Yakuchinone A and Yakuchinone B, and their salts, oxidants, reductants, glycosides and esters thereof (U.S. Patent Application 20030147979; U.S. Pat. No. 5,891,924). Further examples of curcumin analogs include but are not limited to (a) ferulic acid, (i.e., 4-hydroxy-3-methoxycinnamic acid; 3,4-methylenedioxy cinnamic acid; and 3,4-dimethoxycmnamic acid); (b) aromatic ketones (i.e., 4-(4-hydroxy-3-methoxyphenyl)-3-buten-2-one; zingerone; -4-(3,4-methylenedioxyphenyl)-2-butanone; 4-(p-hydroxyphenyl)-3-buten-2-one; 4-hydroxyvalerophenone; 4-liydroxybenzylactone; 4-hydroxybenzophenone; 1,5-bis(4-dimethylaminophenyl)-1,4-pentadien-3-one); (c) aromatic diketones (i.e., 6-hydroxydibenzoylmethane) (d) caffeic acid compounds (i.e., 3, 4-dihydroxycinnamic acid); (e) cinnamic acid; (i) aromatic carboxylic acids (i.e., 3,4-dihydroxyhydrocinnainic acid; 2-hydroxycinnamic acid; 3-hydroxycinnamic acid and 4-liydroxycinnamic acid); (g) aromatic ketocarboxylic acids (i.e., 4-hydroxyphenylpyruvic acid); and (h) aromatic alcohols (i.e., 4-hydroxyphenethyl alcohol). These analogs and other representative analogs that can be used in the present invention are further described in WO95/18606 and WO01/040188. Other known curcumin derivatives and analogs, including dimers, dextran and dendrimer conjugates, may also be used (see, e.g., Raja, K. S., et al. “Synthesis of Novel Curcumin/Tetrahydrocurcumin dimers, monofunctional curcumin/tetrahydrocurcumin analogs and polymers as bioconjugation dyes, for imaging applications, for the treatment of Alzheimer's, prion disease, the treatment of cancer, and as active ingredients in cosmetic formulations,” U.S. Provisional Application No. 60/829,185; Shi, W., et al “Synthesis of mono-functional curcumin derivatives, clicked curcumin dimers and curried cystamine PAMAM dendrimers for imaging and therapeutic applications” Org. Lett. 2007 Dec. 20; 9(26):5461-4).

The curcumin or curcumin derivative may be provided as a conjugate such as a prodrug. Examples of curcumin prodrugs are known (see, e.g., Lu, P., et al. J Huazhong Univ Sci Technolog Med. Sci. 2005; 25(6):668-70, 678, Kapoor, N., et al. Cancer Lett. 2007 Apr. 18; 248(2): pp. 245-50), and methods of making prodrugs are known (see, e.g., WO/2006/076734; U.S. Pat. No. 5,952,294; Balant, L. P., et al. Eur. J. Drug Metab. Pharmacokinet. 1990; 15: pp. 143-153; Bundgaard, H., et al. Drugs of the Future 1991; 16: pp. 443-458). See also U.S. Published Patent Application US 2007/0270464.

The curcumin or curcumin derivative may also be modified to target the active compound to particular cells or tissue by methods known in the art. Examples of tumor-targeted curcumin derivatives (see, e.g., Lu, P., et al. J Huazhong Univ Sci Technolog Med. Sci. 2005; 25(6): pp. 668-70, 678) and telomere-targeted curcumin derivatives (Mishra, S., et al. Nucleic Acids Res Suppl. 2002; (2): pp. 277-8) are known. In some embodiments, curcumin or curcumin derivatives are modified by known methods to target the compounds to the central nervous system (for general methods of targeting the CNS, see Boado, R. J., et al. Biotechnol. Bioneg. 2007; 97: pp. 1376-1386; Pardridge, W. M. Pharm Res. 2007 Jun. 7; [Epub ahead of print, ISSN: 0724-8741 (Print) 1573-904X (Online)]. In particular embodiments, the active compounds are modified to target the brain.

Standard reference works setting forth the general principles of cancer biology known to those of skill in the art include: Holland Frei—CANCER MEDICINE, 7th Ed., (Kufe, D. W. et al., editors), Hamilton (Canada): BC Decker Inc; 2005. Standard reference works setting forth the general principles of neurochemistry known to those of skill in the art include: BASIC NEUROCHEMISTRY, MOLECULAR, CELLULAR, AND MEDICAL ASPECTS, 6th ed. (Siegal, G. J., et al. editors.) Philadelphia: Lippincott, Williams & Wilkins; 1999. Standard reference works setting forth the general principles of medical physiology and pharmacology known to those of skill in the art include: Harrison's PRINCIPLES OF INTERNAL MEDICINE, 14th Ed., (Anthony S. Fauci et al., editors), McGraw-Hill Companies, Inc., 1998.

Throughout this specification and paragraphs, the word “comprise” or variations such as “comprises” or “comprising”, will be understood to imply the inclusion of a stated integer or group of integers but not the exclusion of any other integer or group of integers.

The following are examples which illustrate the compositions and methods of this invention. These examples should not be construed as limiting: the examples are included for the purposes of illustration only. This invention has been described with reference to its preferred embodiments. Variations and modifications of the invention, will be obvious to those skilled in the art from the foregoing detailed description of the invention. It is intended that all of these variations and modifications be included within the scope of this invention.

### EXAMPLES

### Example 1

### Curcumin Formulation

Curcumin has very low solubility in water. To increase its solubility, curcumin was dissolved in 3% DMSO in sterile phosphate buffered saline (PBS). Using this solvent system, a 667 μM solution of curcumin was prepared for injection into subjects. When 200 μl of 667 μM solution is injected into a mouse (approximately 4 ml of body fluid), the final concentration of DMSO is expected to be about 0.15%. For intracranial curcumin injections, curcumin was dissolved in 15% DMSO in sterile PBS to obtain a 3 mM solution. When 5 μl of the 3 mM solution is injected directly into the brain (average volume 400 μl), the final concentration is expected to be 40 μM curcumin and less than 0.2% DMSO.

### Example 2

### The Effect of Curcumin on Tumor Cell Viability In Vitro

Cells. B16F10 (mouse melanoma), GL261 (mouse glioma), HOG (human oligodendroglioma), A549 (human lung cancer), and N18 (mouse neuroblastoma) cells were cultured separately in DMEM containing 10% (v/v) fetal bovine serum and 1% (v/v) penicillin-streptomycin (PS) and allowed to grow to 40% confluence in a 96-well plate.

Curcumin Treatment. Two hours before drug treatment, the medium in each well was replaced with 200 μl Neurobasal medium supplemented with 2% (v/v) B27 and 1% (v/v) PS (Drug-treatment Medium). A stock solution (40 mM) of curcumin was prepared in sterile dimethyl sulfoxide (DMSO) (Example 1). Through serial dilution of each stock solution in Drug-treatment Medium, the following concentrations were obtained: 20 μM and 50 μM (for caspase 3/7 assays) or 25 μM and 50 μM (for MTT assays). Drug-treatment Medium in each well was aspirated, and a drug solution of each concentration or carrier-containing medium (control) was added to triplicate wells of cells (50 μl/well). Cells were incubated with the drugs for 48 h and 96 h in a 37° C. tissue culture incubator. Appropriate control wells were prepared using solutions containing the same volume of DMSO in the same medium but without drug. Additionally, the background absorbance was determined from parallel wells containing only the Drug-treatment Medium plus a particular concentration of the drug (no cells).

Caspase-3/7 Assay. After drug addition, the plates were incubated in a 37° C., humidified CO₂ (5%) incubator for 16 h following which the cells were subjected to caspase3/7 assay using the SensoLyte™ Homogeneous Rh110 Caspase-3/7 Assay Kit (AnaSpec, San Jose, Calif.). First, a dithiothreitol (DTT) solution was prepared mixing 40 μl of 1 M DTT (component E) with 1 ml of assay buffer (component D). Next, 1 μl of caspase-3/7 substrate (component A) was diluted in 100 μl of the DTT solution followed by mixing. The diluted caspase-3/7 substrate solution (50 μl) was added to each well followed by gentle shaking of the plate for 60 min on a shaker set at 100 rpm. Fluorescence in each well was measured using the FLx 800 plate reader (Bio-Tek Instruments, Winooski, Vt.) set at 485/20 nm excitation and 528/20 nm emission wavelengths and the sensitivity of 70. Statistical analysis was performed using ANOVA with Bonferroni post-hoc tests.

Cell Viability Assay Using MTT (3-(4,5-Dimethylthiazolyl-2)-2,5-Diphenyltetrazolium Bromide). Cells were plated and then treated on the next day with drugs in triplicate in 100 μl medium per well (as described above). For curcumin treatment, parallel wells (in triplicate) containing only medium (no cells) and the same concentrations of curcumin were used as background. The cells (2,000/well) were incubated in triplicate in a 96-well plate in the presence or absence of indicated test samples in a final volume of 0.1 ml for specific times required for an experiment. MTT solution (5 mg/ml in PBS) (25 μl) was added to each well. The cells were incubated at 37° C. for 2 h. The extraction buffer (20% SDS, 50% dimethylformamide) (100 μl) was added to each well, the contents gently mixed and incubated overnight at 37° C. Absorbance was measured at 570 nm using a 96-well multi scanner auto-reader with the extraction buffer used as blank. For curcumin samples, the absorbance obtained from the corresponding curcumin plus medium samples was subtracted.

$$Percent cell viability = \frac{OD of the Experimental Sample}{OD of the Control} \times 100$$

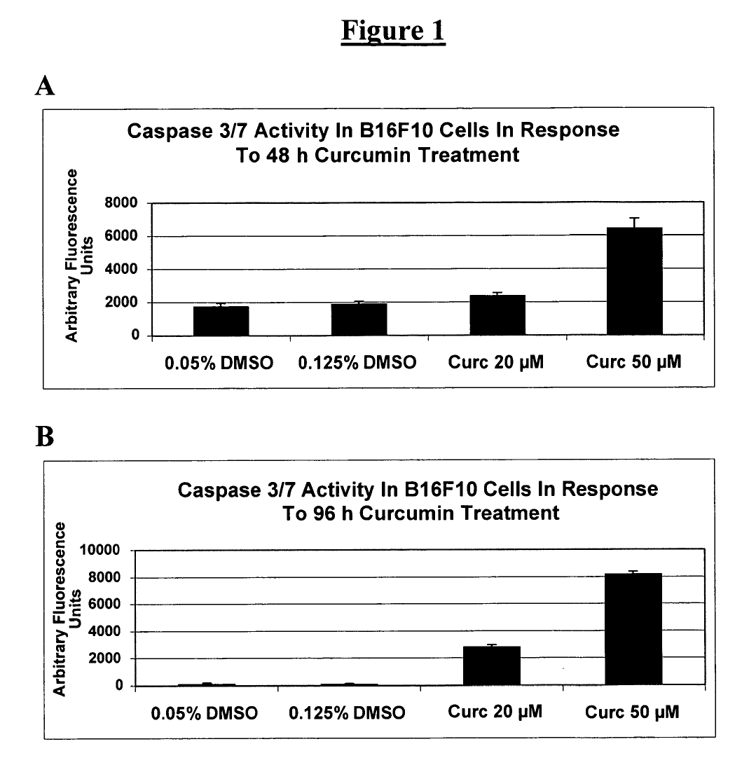

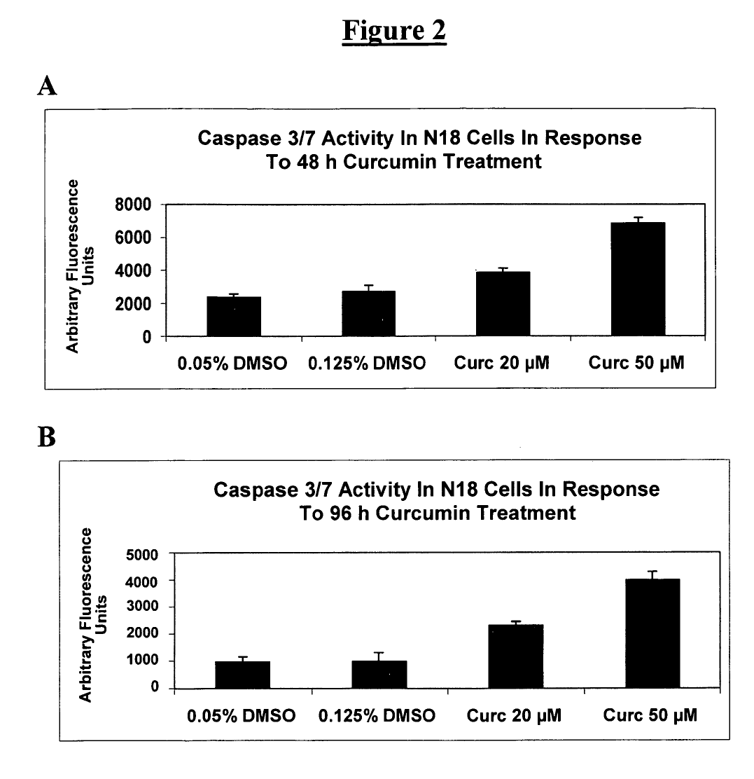

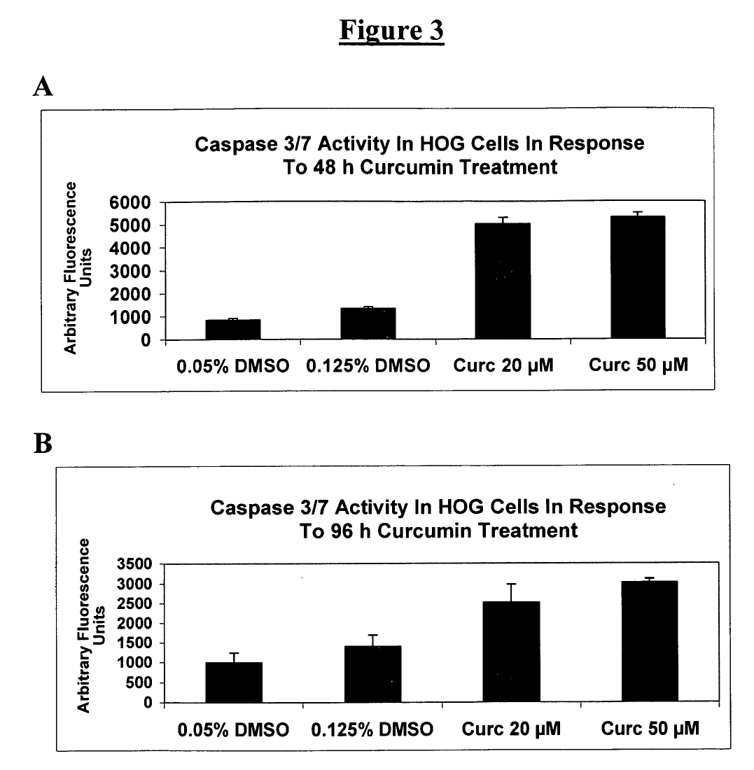

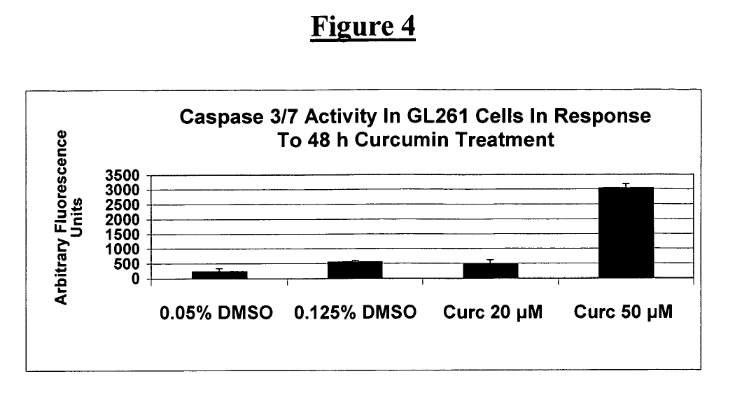

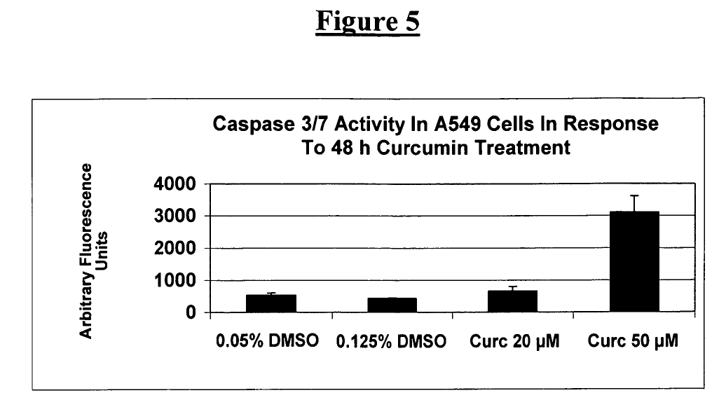

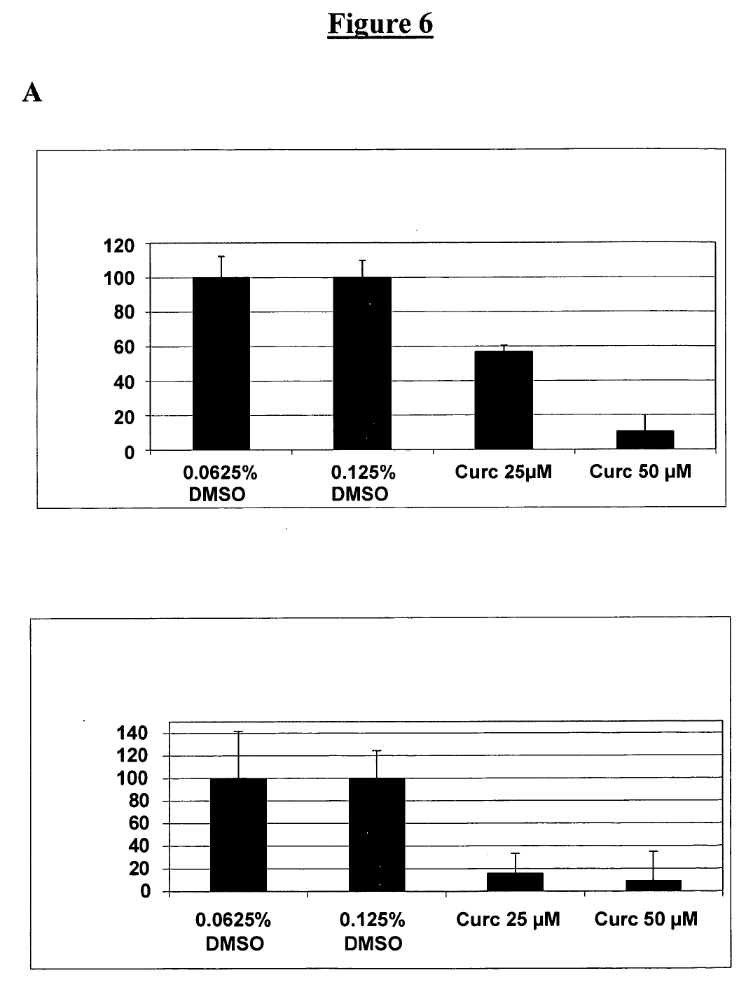

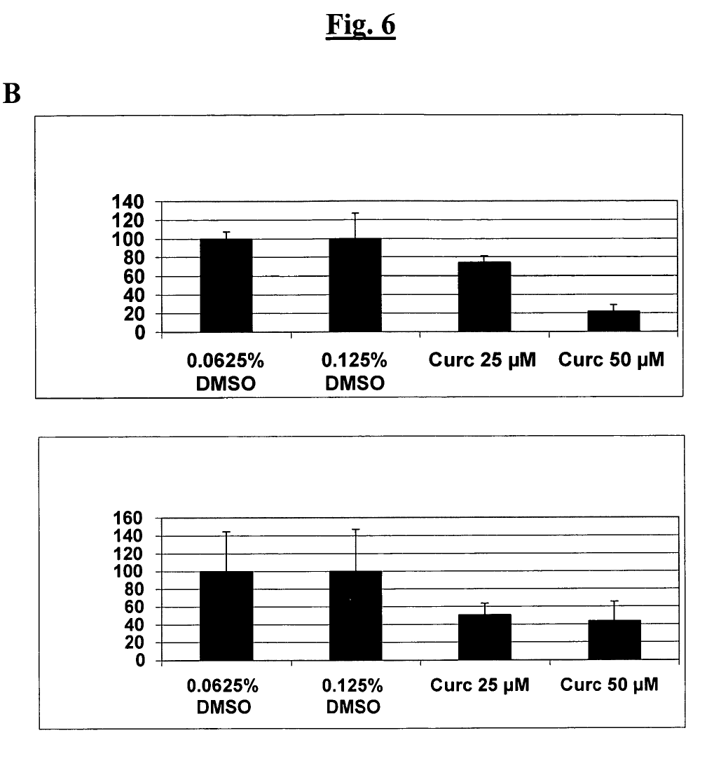

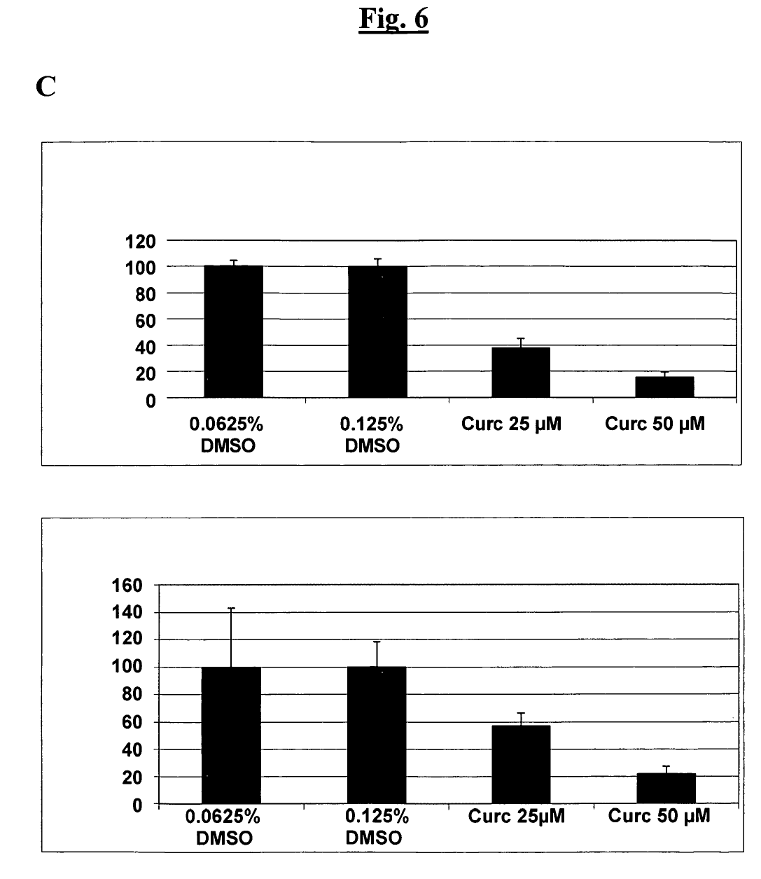

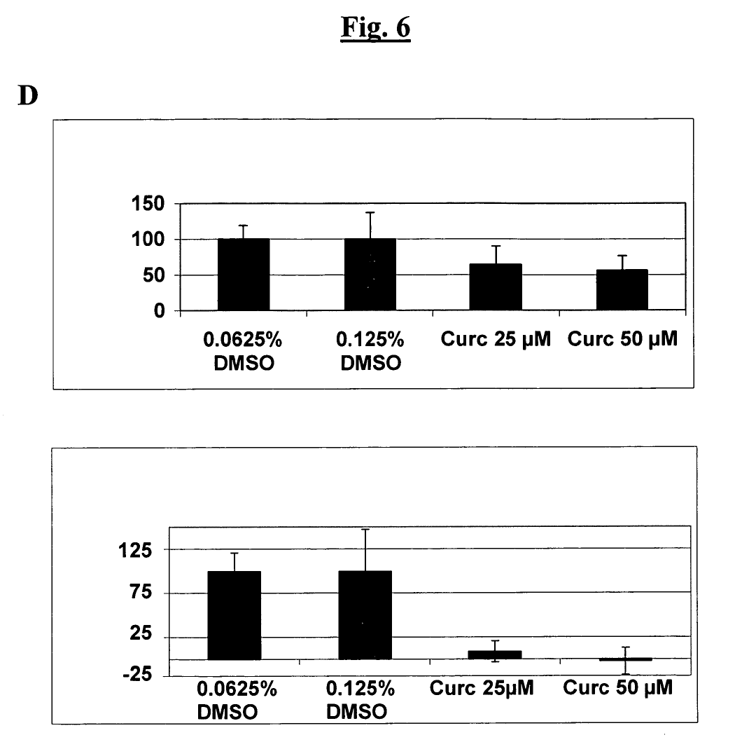

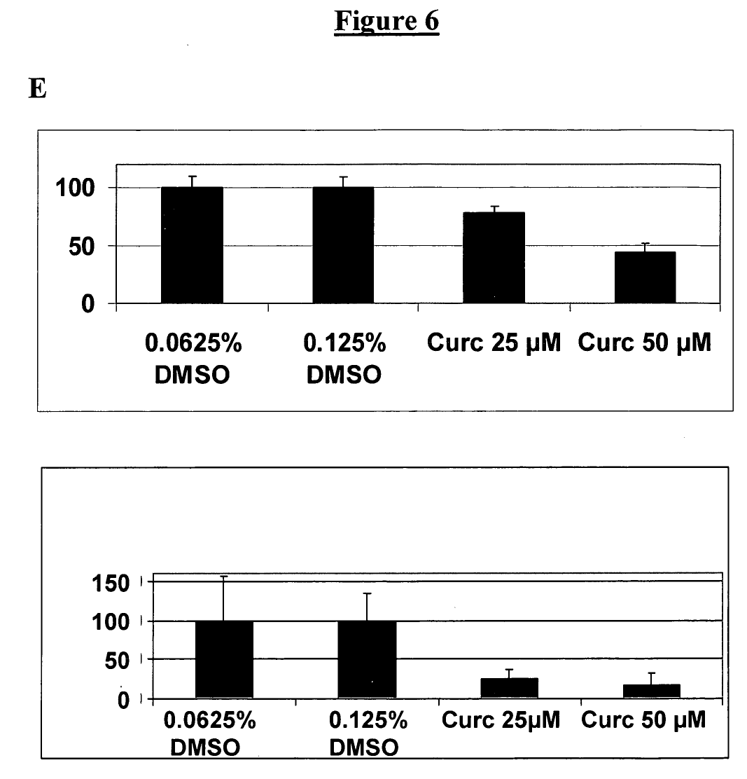

Curcumin treatment increased caspase-3/7 activity (which causes programmed cell death or “apoptosis”) in B16F10 (FIG. 1), N18 (FIG. 2), HOG (FIG. 3), GL261 (FIG. 4), and A549 (FIG. 5) cells. A cell viability assay using MTT also showed a dramatic decrease in viability of the B16 F10 (FIG. 6a), GL261 (FIG. 6b), N18 (FIG. 6c), A549 (FIG. 6d) and HOG (FIG. 6e) cells in the presence of curcumin compared to control cells that were not treated with curcumin.

### Example 3

### Testing the Permeability of the Blood-Brain-Barrier to Curcumin

A curcumin solution (200 μl of 667 μM curcumin in PBS containing 3% DMSO) was injected through the tail vein of each mouse, the mice were sacrificed after 15 min, 30 min, and 2 hours, and brain regions (forebrain, hippocampus, and hypothalamus) as well as blood were collected for analysis. Each tissue fraction was diluted into 300 μl of water, homogenized, and then diluted with 700 μl of acetonitrile. Proteins and other insoluble substances were separated by centrifugation at 8000 rpm in a table-top Eppendorf microcentrifuge, and the supernatants were transferred to fresh tubes and then evaporated by blowing in nitrogen gas. The blood (100 μl) was diluted with 40 μl EDTA (500 mM) and 160 μl of water. This mixture was diluted with 700 μl of actonitrile, vortexed, protein and debris separated by centrifugation and then the supernatant evaporated under nitrogen. The residues obtained were dissolved in 50 μl of acetonitrile:water (70:30) and 30 μl of this solution injected for HPLC analysis on a C18 reverse-phase column. A standard 0.1 mM solution of curcumin dissolved in acetonitrile:water (70:30) was injected for comparison. The mobile phase was a 30-70% gradient of acetonitrile in water containing 0.1% trifluoroacetic acid (TFA). Curcumin eluted as a closely spaced triplet with the strongest peak eluting at 12.8 min.

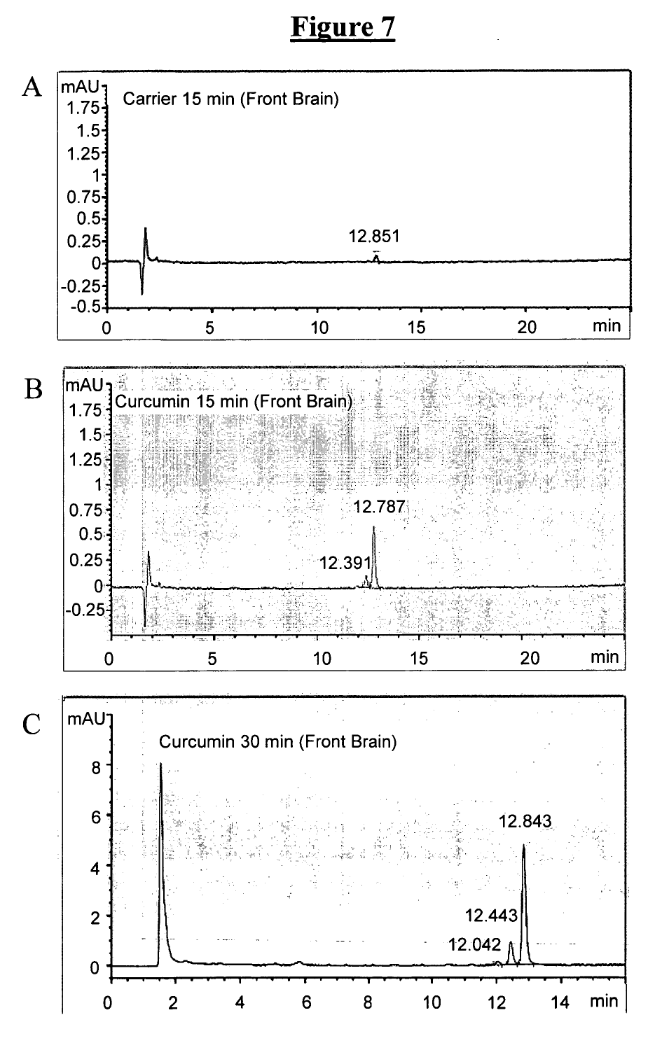

The HPLC traces showed the presence of curcumin in mouse brain within 15 minutes (FIG. 7b) and 30 minutes (FIG. 7c) of tail vein injection. The curcumin was completely metabolized within 2 hours of injection (FIG. 7d). No curcumin was observed in the HPLC traces of carrier injected mouse brains within 15 minutes (FIG. 7a).

### Example 4

### The Effect of Curcumin on Tumor Formation in Peripheral Tissue

Generation of Tumors. C57BL mice were given food and water ad libitum and subjected to 12 hour dark/12 hour light cycles. To generate tumors in the peripheral tissue, B16F10 mouse melanoma cells (10⁴ cells in 10 μl PBS) were injected into the necks of male and female C57BL mice.

Curcumin Treatment. The control mice received, aseptically, the carrier (3% DMSO in 200 μl PBS) daily through the tail vein after placing the mouse in a restraining cage without anesthetics. Other mice similarly received 667 curcumin in 200 μl of PBS containing 3% DMSO daily through the tail vein.

The carrier-injected mice developed either a tumor (the male mice) or a lesion around the site of injection (female mice), lost weight, and died in about a month. In sharp contrast, the curcumin-injected mice (male and female) showed no outward sign of tumor or lesion and remained normal indefinitely even after the curcumin injection had been discontinued for 18 days.

### Example 5

### Effect of Curcumin on Brain Tumor Cells

Generation of Tumors. The mice were given food and water ad libitum and subjected to 12 hour dark/12 hour light cycles. To generate brain tumors in mice, B16F10 mouse melanoma cells or GL261 mouse glioma cells (1000 cells in 5 μl PBS per C57BL mouse) were injected into the right front brain [coordinates: with respect to the Bregma (in mm) AP=2.5; L=−1.1; D=1.5] at the rate of 1 μl per minute using a stereotaxic set-up (KDS Model 310plus infusion-withdrawal syringe pump) (Paxinos, G., and Franklin, K. B. J., The mouse brain in stereotaxic coordinates. 2nd ed. 2001, New York: Academic Press). Before the cell injection, the mouse was anesthetized by injecting (sc) xylazine (10 mg/Kg) and ketamine (100 mg/Kg), the head was shaved, and then the head was immobilized on a Stoelting sterotaxic frame. The mouse was determined to be in deep anesthesia by checking for regular, relaxed respiration and the lack of response to tail/toe pinch before its head was immobilized. The head was cleaned with 70% ethanol and then, using a sterile scalpel, a midline incision was made and subcutaneous muscle and fascia retracted to expose the skull. A hole was made in the skull with a dental drill and the cells injected as described above using a Hamilton syringe fitted to the syringe pump. Under the described condition of anesthesia, the mouse remains unconscious for about 90 min. The total time taken for immobilization of the mouse, exposure of the skull, and injection was about 30 min. Thus, the total time required from the injection of anesthetics to the completion of surgery was less than one hour. After the injection, the hole in the skull was sealed with sterile bone wax and the skin and muscle placed back in place with appropriate 7-mm stainless steel clips (Reflex Skin closure system; Cellpoint Scientific). For wound healing, a topical antibiotic (Triple Antibiotic, containing Polymyxin B, Bacitracin, and Neomycin) was applied and for post-operative care the animal was kept in a warm blanket at 35-37° C. taking precautions to avoid thermal injury. On the first day after surgery (day 2), a sterile, 37° C. solution of physiologic saline was administered (IP) at the dose of 1 ml/100 g body weight. The animals were then caged individually with access to food and water and kept under close monitoring. The mice also received acetaminophen (Tylenol syrup) 1 mg/ml in drinking water daily until they returned to complete normalcy in terms of both behavior and appearance (body weight; reduced locomotion, vocalization, lack of grooming, wound scratching; and signs inflammation at the surgical site).

Curcumin Treatment. The control mice received, aseptically, the carrier (3% DMSO in 200 μl PBS) daily from the next day (day 2), through the tail vein after placing the mouse in a restraining cage without anesthetics. Other mice similarly received 667 μM curcumin in 200 μl of PBS containing 3% DMSO daily from the next day (day 2) for 18 days (through day 19) through the tail vein. Other mice received the same curcumin solution daily from day 4, day 6, day 8 or day 10. One mouse (designated Mouse 2) received a single dose of curcumin (5 μl of 667 μM solution) five minutes after injection of B16F10 cells into the brain, using the same stereotaxic set-up.

Monitoring and Sample Preparation. The effects of brain tumor on movement generally appeared in about two to three weeks. The mice were monitored for disorders in movement using an open field VIDEO monitoring set up (10 min per mouse). Blood was collected from the saphenous vein to test for markers for cancer and cell and organ damage. Additionally, MRI was performed on the mice to monitor the growth and progression of brain tumors. To this end, the animals were anesthetized with a mixture of gaseous isofluorane and oxygen and mounted on a set-up attached to heartbeat and blood pressure monitors and maintained under inhalation of regulated concentrations of isofluorane and oxygen during the entire procedure of MRI recording (approximately 60-90 minutes). After MRI, the proportion of oxygen:isofluorane was slowly increased to attain ambient conditions before transferring each mouse back to its original cage to wake up. The mice were then kept under close observation until they reached behavioral normalcy, as described above.

In addition to MRI screening, the progression of tumor growth was monitored by histological staining (described below) after sacrificing duplicate animals that had been injected with the cells. Mice were sacrificed on days 15, 20, and 25 considering the day of injection of cells as day 1. When the carrier-injected animals appeared to be losing locomotion, they were sacrificed before they died to assess the progression of tumor by pathological analysis. The unaffected animals (curcumin-injected) were observed until day 40 and then sacrificed for pathological analysis as described below.

When the impaired mice were about to die (when they lie on their sides unable to move or feed themselves), the brain of each animal was removed, fixed in paraformaldehyde, soaked in a sucrose solution and sectioned for pathological analysis by hematoxylin-eosin (H&E) staining to identify the tumor cells. Some of the brains were used to determine the expression of tumor markers (PV-1, endosialin, and prostaglandin D synthase) (Carson-Walter, E. B. et al., Clin Cancer Res, 2005. 11(21): p. 7643-7650; Brady, J. et al., J. Neuropathol. Exp. Neurol., 2004. 63: p. 1274-1283; Saso, L. et al., Biochem. Mol. Biol. Int., 1998. 46: p. 643-656).

H&E Staining: Sections were soaked in Harris hematoxylin (e.g., Anatech Lmtd. Cat #842) for 2 min and then washed with water ten times (2 min per wash), then once with acid alcohol, once with water, with ammonia solution-10 dips (changes the stain from purple to blue), with running water for 5 min, and, finally, once with 80% EtOH. Next the sections were stained in Eosin solution (e.g., Anatech Cat #837) for 5 min, followed by two washes with 95% ethanol, one wash with 100% ethanol, and two washes (3 min per wash) with xylene. Xylene was removed by wiping the back and tipping the slide, and the stained section were mounted in Permount.

Injected curcumin blocked brain tumor formation and morbidity in mice injected with B16F10 cells in the right frontal lobe. The mouse receiving daily carrier injections (“carrier-injected mouse”) displayed movement disorders on day 20 coupled with seizures lasting almost 30 seconds, whereas the mouse receiving daily curcumin injections (“curcumin injected mouse”) showed normal movement. By day 21, the carrier-injected mouse lay on its side, breathing heavily, but unable to move or feed itself (it was sacrificed and its brain fixed for histology), whereas the curcumin-injected mouse displayed normal movement and behavior on days 21, 28, and 39. The mouse that received a single dose of curcumin (Mouse 2) was hunched up and morbid on day 21. Large tumors were observed in the right frontal lobe of both the carrier-injected mouse and the mouse that received a single dose of curcumin (Mouse 2), but not the curcumin-injected mouse.

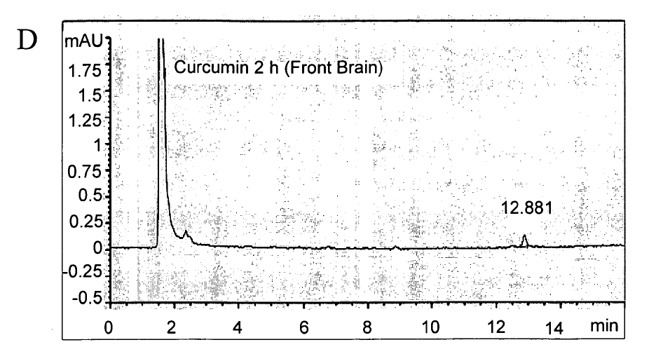

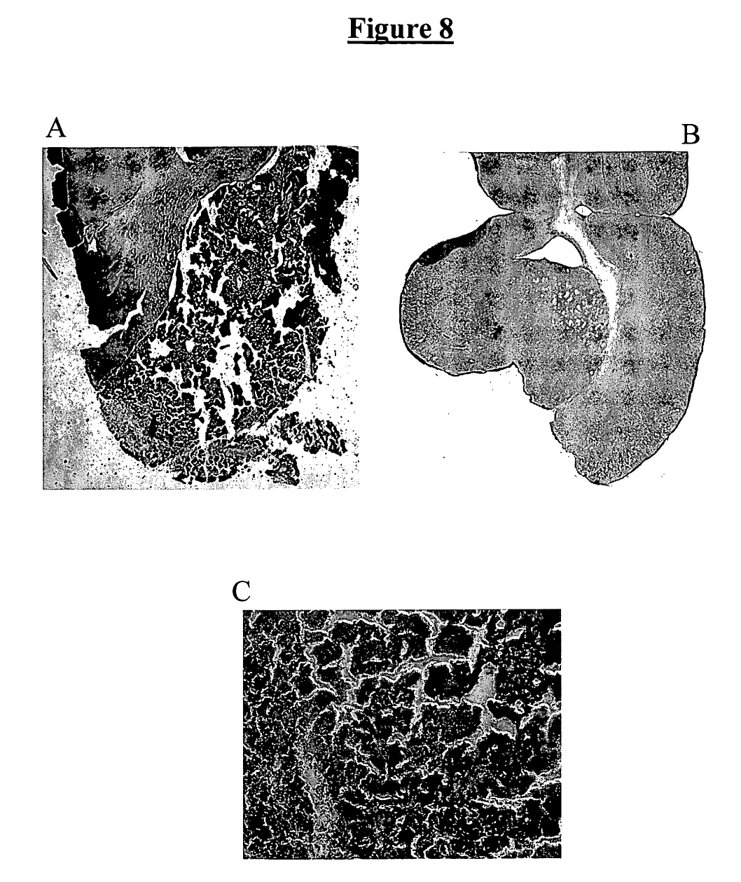

Hematoxylin and eosin staining of brain tissue revealed massive tumor and tissue damage in the control mouse (FIGS. 8A and 8C). This tissue disintegration was completely blocked in the curcumin-injected mice (FIG. 8B).

### Example 6

### The Effect of Curcumin on Signaling Pathways Involved in Tumor Cell Viability

B16F10 cells were cultured in 10-cm plates and then treated in duplicate with carrier or curcumin (50 μM). Following treatment for appropriate time periods, the cells were washed with PBS, lysed in RIPA buffer (PBS containing 1% Nonidet P40, 0.5% sodium deoxycholate, 0.1% SDS, 0.5 mM Na₃VO₄ plus freshly added PMSF and protease inhibitor cocktail; Boeringer-Roche), and then 20 μg of lysate protein (per lane) were analyzed by SDS-10% PAGE and Western blot analysis. Antibody concentrations used were: anti-phospho-NF-κB (1:1000) (Santa Cruz Biotechnology, Santa Cruz, Calif.), anti-phospho-Akt (1:5000) (Cell Signaling, CA), anti-cyclin D1 (1:1000) (Cell Signaling, CA), anti-Bcl_{XL} (1:500) (Santa Cruz Biotechnology), anti-VEGF (1:500) (Santa Cruz Biotechnology), and anti-β-actin (1:1000) (Sigma, St. Louis, Mo.).

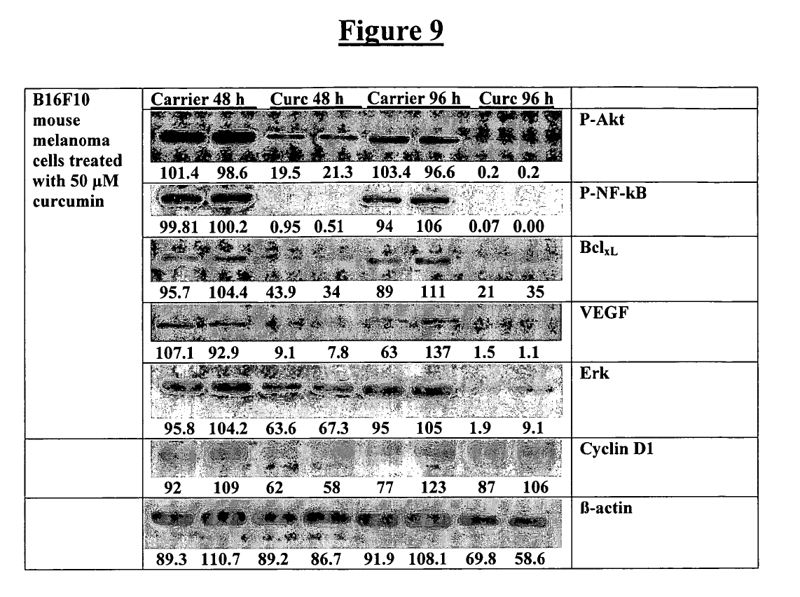

Western blot analysis showed that curcumin treatment of cultured B16F10 cells caused suppression of markers of cell viability, including cyclin D1 as well as phospho-NF-κB, phospho-Akt, Erk, and Bcl_{XL} (FIG. 9). Intriguingly, during active cell division (first 48 h), curcumin caused a decrease in cyclin D1 levels, but subsequently, when cell division had slowed down because of confluency and cell death, cyclin D1 inhibition was not observed in the curcumin-treated cells. Furthermore, the angiogenesis- and tumorigenesis-associated protein VEGF was also suppressed following curcumin treatment.

### Example 7

### The Effect of Curcumin on Normal Brain Cell Viability In Vivo

In each experiment, three mice were injected with 200 μl of PBS containing 3% DMSO, and three mice were injected with 200 μl of 667 μM curcumin (as described earlier) through the tail vein on three consecutive days by the method described above. On the fourth day, the mice were perfused with PBS through the heart, and the brains sectioned into 400-μm sections. Similar brain sections were also made from untreated mice which were then treated with 1 mM H₂O₂ in PBS for one hour, followed by incubation in H₂O₂-free DTM for three hours. These samples were used as a positive control for cell death.

The sections from each mouse were placed in 400 μl PBS in one well of a 48-well plate (i.e. three wells for three mice) and then treated with 200 μl of MTT (5 mg/ml) per well with gentle mixing at 37° C. for about 2 hours. Subsequently, 800 μl of the lysis buffer was added to each well, and the plate was sealed and incubated overnight at 37° C. with gentle mixing. Three 100-μl portions of the tissue solution obtained were next transferred to three respective wells of a 96-well plate (i.e. three wells per mouse and nine wells for three mice in one group) and absorbance at 570 nm was measured using a 96-well multi scanner auto-reader. Results obtained were normalized to protein levels and then expressed as percent of carrier-treated samples.

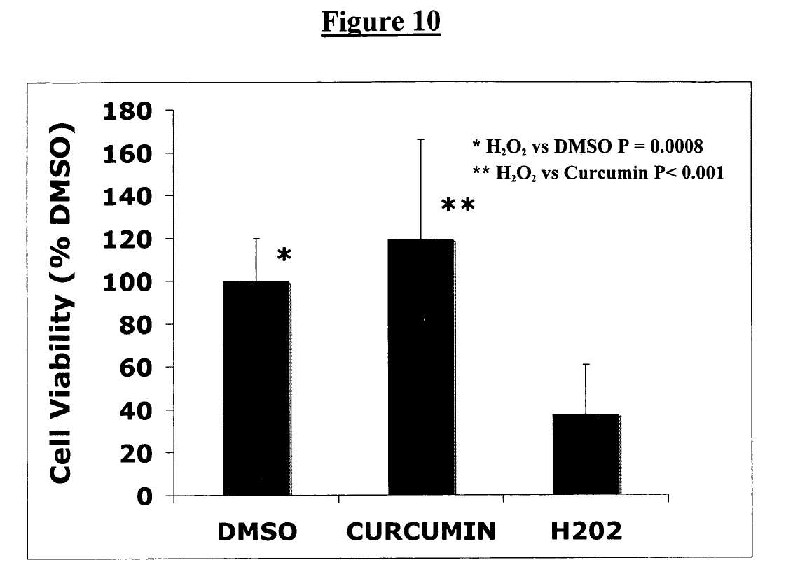

Curcumin treatment did not cause suppression of cell viability in the brain when compared to the carrier-injected mice, whereas the H₂O₂-treated brain slices showed a dramatic decrease in cell viability as measured by MTT activity (FIG. 10).

### Example 8

### The Effect of Intracranial Injection of Curcumin on Brain Tumor Cells

Each mouse was fitted with a stainless steel guide cannula (Plastic Ones, System C313G, Roanoke, Va., USA) to administer curcumin directly to the brain. Cannulas were implanted into the right frontal cortex at the same coordinates as used earlier for intracerebral injection. The guide cannula was fixed to the skull with dental cement. A removable stylet plugged the guide cannula except during the time of drug injection. B16F10 cells (103) were delivered into mice in two sets on the first day. Starting from the third day, 5 μl of curcumin (3 mM) in PBS containing 15% DMSO were infused through the cannula of one set of mice (three), whereas the other set of mice (three) received 5 μl of PBS containing 15% DMSO (carrier). Considering that the brain volume of an adult mouse is about 400 μl, dispersion of this solution would give a final concentration of 37.5 μM curcumin and 0.187% DMSO in the brain. This infusion was started on the third day and then performed every other day, until day 19. The brains of the treated mice were removed and soaked (fixed) in 4% paraformaldehyde.

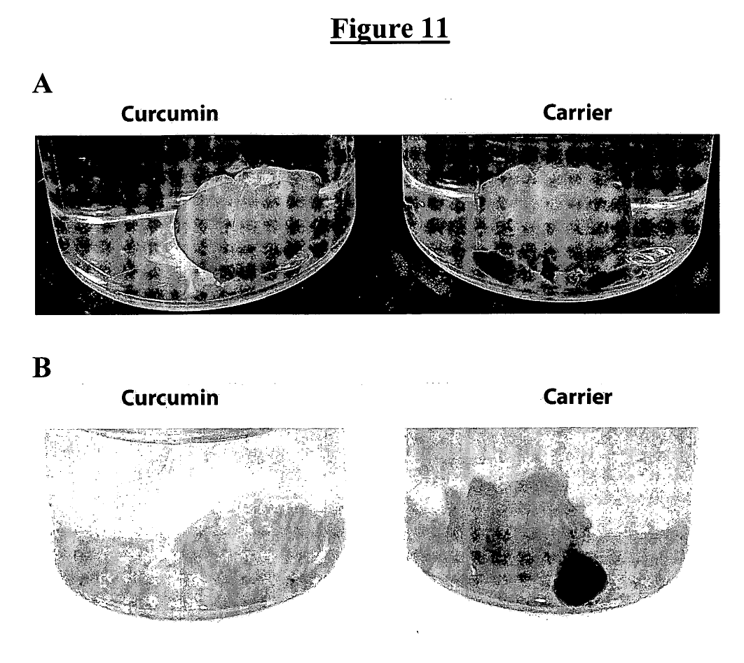

Mice receiving the carrier generally collapsed between day 15-25, whereas the curcumin-infused mice showed normal behavior. None of the curcumin-treated mice had a detectable brain tumor, whereas the carrier-treated mice showed sizeable brain tumors (FIG. 11). The dark cancerous tissue containing melanin-producing B16F10 cells was often so fragile that it broke loose from the rest of the brain.

1.-13. (canceled)

14. A method for diminishing cancer cell growth; decreasing tumor size; preventing tumor formation; preventing cancer or tumor cell invasion or metastasis in a tissue; preventing or inhibiting the recurrence of tumors or diminishing the side effects after surgery, radiation or chemotherapy; improving cancer patient prognosis; increasing remission or survival time; or decreasing angiogenesis in a subject comprising the step of administering to the subject a composition comprising a curcumin compound.

15. The method of claim 14, wherein the cancer cell is associated with a tumor of the nervous system.

16. The method of claim 15, wherein the cancer cell or tumor is selected from the group consisting of glioma, metastases, meningioma, pituitary adenoma and acoustic neuroma.

17. The method of claim 15, wherein the tumor is selected from astrocytoma, pilocytic astrocytoma, low-grade astrocytoma, anaplastic astrocytoma, glioblastoma multiforme, brain stem glioma, ependymoma, subependymoma, ganglioneuroma, mixed glioma, oligodendroglioma, optic nerve glioma, acoustic neuroma, chordoma, eNS lymphoma, craniopharyngioma, emangioblastoma, medulloblastoma, meningioma, pineal tumors, pituitary tumors, primitive neuroectodermal tumors (PNET), rhabdoid tumors, schwannoma, gliomas of the optic nerve, neurofibromas of 8th cranial nerve, neurofibromas of 5th cranial nerve, arachnoid, dermoid, epidermoid, colloid and euroepithelial cysts.

18. The method of claim 17, wherein the tumor is a metastasis from a primary tumor.

19. The method of claim 18, wherein the metastasis is from a primary tumor of the lung, skin (melanoma), kidney, colon or breast.

20. The method claim 14, wherein the subject is a human patient in need of treatment.

21. The method of claim 14, wherein the curcumin compound is curcumin or an analog or derivative of curcumin having increased solubility in aqueous solution.

22. The method of claim 21, wherein the resulting plasma concentration of curcumin compound is 5-100 μM.

23. The method of claim 14, wherein the composition is administered intravenously.

24. The method of claim 14, wherein the composition further comprises DMSO.

25. The method of claim 14, wherein the composition further comprises a factor selected from the group consisting of a second chemotherapeutic agent, a diagnostic agent, an anti-oxidant, an anti-inflammatory, a growth factor, a hormone or a nutrient.

26. The method of claim 14, wherein the composition is administered by a prolonged treatment.
